(19) United States
(12) Patent Application Publication
(10) Pub. No.: US 2022/0226672 A1
(43) Pub. Date: Jul. 21, 2022

(54) FOCUSED ULTRASOUND DEVICE AND METHOD FOR DERMATOLOGICAL TREATMENT

(71) Applicant: NATIONAL HEALTH RESEARCH INSTITUTES, Miaoli County (TW)

(72) Inventors: GIN-SHIN CHEN, Miaoli County (TW); FENG-HUEI LIN, Miaoli County (TW); JIUN-JUNG CHEN, Miaoli County (TW); CHIA-CHING PAN, Miaoli County (TW)

(73) Assignee: NATIONAL HEALTH RESEARCH INSTITUTES, Miaoli County (TW)

(21) Appl. No.: 17/614,532

(22) PCT Filed: Jun. 10, 2019
(86) PCT No.: PCT/US19/36415

§ 371 Date: Nov. 28, 2021

(51) Int. Cl. A61N 7/02; B06B 1/06

(52) U.S. Cl. CPC A61N 7/02; A61N 2007/0008; B06B 1/0622

(57) ABSTRACT

A focused ultrasound device and method for dermatological treatment. The focused ultrasound device includes a focused ultrasound applicator, an automatic motion mechanism, a driver, a computer, a software operation interface. In addition, the focused ultrasound method for dermatological treatment utilizes the same focused ultrasound applicator, to achieve coaxial deep layer and shallow layer ablation treatment of the skin.

### BACKGROUND OF THE INVENTION

### Field of the Invention

The present invention relates to an ultrasound device and method, and in particular to a focused ultrasound device and method for dermatological treatment.

### The Prior Arts

Nowadays, focused ultrasound dermatological treatment is used extensively in treatment of tattoo, varicose veins, hyperhidrosis, and face lift, etc. The principle of focused ultrasound dermatological treatment is that, ultrasound is produced outside the body, that is transmitted through a medium to focus on the target tissues. In the focal zone, the temperature of the tissue is raised. The highest temperature and the temperature increase duration required depend on various parameters of ultrasound. In this respect, the burning/ablation of subcutaneous tissue is taken as an example for explanation. In this case, the focal zone intensity must be greater than 1000 W/cm², operation duration is less than 2 seconds, and the temperature is about 60° C. And this could cause denaturation of collagen, and then remolding of collagen. As such, the focused ultrasound is able to realize high precision and noninvasive treatment.

Presently, the focused ultrasound device for dermatological treatment is equipped with focused ultrasound applicators of two or three different specifications, to achieve subcutaneous tissue ablation at different depths. In other words, a specific focused ultrasound applicator must be used to realize tissue ablation at a specific depth. When it is required to treat the tissue at a different depth, the focused ultrasound applicator must be detached and replaced with another focused ultrasound applicator. In order to eliminate the inconvenience of attaching and detaching, and to save time of treatment, the following Patents are proposed: U.S. Pat. No. 9,833,640B2 “Method and System for ultrasound treatment of skin”; U.S. Pat. No. 9,895,560B2 “Method for rejuvenating skin by heating tissue for cosmetic treatment of face and body”. In which, an annular phased array focused ultrasound applicator is used to realize the subcutaneous tissue ablations at different depths. For the annular phased array focused ultrasound applicator, 8-16 piezoelectric elements are provided therein. The geometry structure of the applicator is of a bowl shape piezoelectric element, and its surface is divided into 8-16 annular electrodes. The annular electrode is defined as a piezoelectric element. Its principle of operation is to regulate and control the electrical phase of the signal input to the array piezoelectric element, so that the ultrasound waves emitted by the various piezoelectric elements are converged to a predetermined position to form a focal zone, to achieve dynamic focusing of the annular phased array focused ultrasound applicator along its axial direction. In other words, it could realize the deep and shallow movements of the focal zone along the axial direction of the applicator, without the need of moving or replacing the applicator.

However, the shortcomings of this design are that, in the treatment of the skin of a face, the curves in the facial contour could limit the contact area between the focused ultrasound applicator and the skin, this in turn limits the size of the acoustic window. In this regard, the acoustic window defines the area the ultrasound may enter into the skin. As such, due to the limited size of the acoustic window, the annular phased array ultrasound applicator can not produce sufficient energy and heat for the focal area (>1000 W/cm²) at different depths. The geometric limitation and the focal zone high heat requirement result in the consequence that the annular phased array focused ultrasound applicator is not able to perform dynamic focusing to achieve facial skin treatment. In addition, the existing phased array applicator has other drawbacks that, its focusing is rather unstable and its cost is high, compared with the non-phased array ultrasound applicator.

Therefore, presently, the design and performance of the focused ultrasound device for dermatological treatment is not quite satisfactory, and it leaves much room for improvements.

### SUMMARY OF THE INVENTION

In view of the problems and drawbacks of the prior art, the present invention provides a focused ultrasound device for dermatological treatment, to overcome the deficiency of the existing technology.

The major objective of the present invention is to provide a focused ultrasound device and method for dermatological treatment. In the present invention, a plurality of transducers are placed into an applicator, to realize localized ablations of deep and shallow tissues along the axial direction of the applicator, through the geometric structure and the specific arrangement of the transducers, and by using the specific sequence of driving different transducers. In this way, the plurality of transducers can be placed into a cassette type applicator, such that the transducer is able to achieve scanning type ablation treatment, by using an automatic motion mechanism.

In the present invention, two or three different focusing depths are achieved by making use of the geometric structure and special arrangement of the plurality of transducers. In the following, the case of using two kinds of transducers (including a central transducer and two side transducers) is taken as an example for explanation, yet that can be applied similarly to the design using three kinds of transducers.

In order to achieve the objectives mentioned above, the present invention provides a focused ultrasound device for dermatological treatment, comprising: a focused ultrasound applicator, an automatic motion mechanism, a driver, a computer, and a software operation interface.

Wherein, the focused ultrasound applicator is of a cassette type package structure, including: an ultrasound transducer, a transducer packaging structure, a transmission axle, a sealing film disposed at a front opening of the cassette type package structure, a tenon A , a mortise B, a tenon C, a mortise D, a water proof separation layer, a transducer cassette upper shell, a transducer cassette middle shell, and a transducer cassette lower shell. The ultrasound transducer is disposed in the transducer packaging structure, and the transducer packaging structure is movable linearly along the transmission axle. A tenon A is disposed at a top end of the transducer packaging structure, and is connected to a mortise B on the transmission axle of a step motor. The sealing film disposed at a front opening of the cassette type package structure is adapted to work in cooperation with the water proof separation layer, to tightly seal off water in the cassette type package structure, to serve as a transmission medium of the ultrasound.

The central transducer has geometric structure of a concave shell body symmetric in left and right directions, and in up and down directions. The geometric parameters of the central transducer are as follows: a curvature radius R1, a bend polar angle θ1, a bend azimuth angle φ1, a shell thickness t1, an outer width W1, an inner width W2, an outer length L1, an inner length L2. As shown in FIG. 6, in an embodiment of the present invention, the ranges of geometric parameters for the central transducer are as follows: the curvature radius R 1: 10-20 mm, the bend polar angle θ1: 15°-45°, the bend azimuth angle φ1: 80°-120°, the shell thickness t1: 0.2 mm-0.5 mm, the outer width W1: 2 mm-7 mm, the inner width W2: 2 mm -7 mm, the outer length L1: 18 mm-22 mm, the inner length L2: 18 mm-22 mm.

In addition, the side transducer has geometric structure of a concave shell body symmetric in left and right directions. The geometric parameters of the side transducer are as follows: a curvature radius R2, a bend polar angle θ2, a bend azimuth angle φ2, a shell thickness t2, an outer width W3, an inner width W4, an upper outer length L3, an upper inner length L4, a lower outer length L5, and a lower inner length L6. As shown in FIG. 7, in an embodiment of the present invention, the ranges of the geometric parameters for the side transducer are selected as follows: the curvature radius R 2: 10-20 mm, the bend polar angle θ2: 30°-50°, the bend azimuth angle θ2: 70°-120°, the shell thickness t2: 0.2 mm-0.7 mm, the outer width W3: 6 mm-10 mm, the inner width W4: 6 mm-10 mm, the upper outer length L3: 10 mm-13 mm, the upper inner length L4: 10 mm-13 mm, the lower outer length L5: 16 mm-20 mm, and the lower inner length L6: 16 mm-20 mm. The ultrasound transducer is made of piezoelectric material or piezo-composite material.

In the descriptions above, the geometric parameters of the central transducer are chosen as follows: R 1: 13.5 mm, θ1: 17°, φ1: 95°, t1: 0.3 mm, W1: 2.8 mm, W2: 2.7 mm, L1: 20.5 mm, and L2: 20 mm

In the descriptions above, the geometric parameters of the side transducers are chosen as follows: R 2: 15 mm, θ2: 47°, φ2: 73°, 2: 0.5 mm, W3: 10 mm, W4: 9.6 mm, L3: 11.4 mm, L4: 11 mm, L5: 18 mm, and L6: 17.8 mm.

Moreover, the present invention also provides a focused ultrasound method for dermatological treatment, and that utilizes the focused ultrasound device for dermatological treatment mentioned above to implement. Wherein, a same focused ultrasound applicator is used to perform scanning type ablation coaxially for deep layer and shallow layer skin tissues. The method comprises the following steps:

(1) utilize the driver to drive the side transducers in the focused ultrasound applicator, to perform ablation of deep layer target tissues D₁;

(2) shut down the driver, then activate and utilize the driver to drive the central transducer, to perform coaxial ablation of shallow layer target tissues S₁;

(3) utilize the automatic motion mechanism to bring the focused ultrasound applicator to a next treatment position, to repeat driving and ablating of steps 1 and 2 above, to realize ablation of the deep layer target tissues D₂ and the shallow layer target tissues S₂; and

(4) repeat the steps 1 to 3, to perform in sequence driving, motion, and ablation, to realize coaxial ablation of deep layer target tissues D₃ . . . D_{N} and shallow layer target tissues S₃ . . . S_{N}.

Further, the focused ultrasound method for dermatological treatment can also be implemented in the following steps:

(1) utilize the driver to drive the side transducers to act, and control the automatic motion mechanism to move, to perform scanning ablation of deep layer target tissues D₁, D₂, D₃, . . . D_{N}; and

(2) shut down the driver, and then activate and utilize the driver to drive the central transducer, to perform scanning ablation of the shallow layer target tissues S₁, S₂, S₃, . . . S_{N}, to realize coaxial ablation of skin.

Through using the focused ultrasound device and method for dermatological treatment, the deficiencies and drawbacks of the existing technology can be avoided. Namely, the drawback of the existing technology is that, due to geometric limitation and the focal zone high heat requirement, the annular phased array focused ultrasound applicator is not able to perform dynamic focusing to achieve facial skin treatment effectively. In contrast, in the present invention, through the geometric structure design, the transducer is designed into a central transducer and two side transducers, such that they are able to produce focal zones at different depths along the same axis of the focused ultrasound applicator to realize ablation, in achieving skin treatment of the various parts, including the facial part of the body effectively.

Further scope of the applicability of the present invention will become apparent from the detailed descriptions given hereinafter. However, it should be understood that the detailed descriptions and specific examples, while indicating preferred embodiments of the present invention, are given by way of illustration only, since various changes and modifications within the spirit and scope of the present invention will become apparent to those skilled in the art from the detailed descriptions.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The related drawings in connection with the detailed descriptions of the present invention to be made later are described briefly as follows, in which:

FIG. 1 is a schematic diagram of a focused ultrasound device for dermatological treatment according to the present invention;

FIG. 2 is a schematic diagram of a focused ultrasound applicator according to the present invention;

FIG. 3 is a schematic diagram of an automatic motion mechanism according the present invention;

FIG. 4 is a schematic diagram of a focused ultrasound applicator and an automatic motion mechanism before and after assembly according to the present invention;

FIG. 5 is a schematic diagram of a focused ultrasound device for dermatological treatment having a central transducer and two side transducers according to the present invention;

FIG. 6 is an exploded view of a central transducer according to the present invention;

FIG. 7 is an exploded view of a side transducer according to the present invention;

FIG. 8 is a schematic diagram showing numerical model of the central transducer and the side transducers according to the present invention;

FIG. 9 is a diagram of simulation results of focal zone for ultrasound focusing at 3 mm under skin according to the present invention;

FIG. 10 is a diagram of simulation results of focal zone for ultrasound focusing at 4.5 mm under skin according to the present invention;

FIG. 11A is a schematic diagram of a focused ultrasound device used in a scanning type pork ablation experiment according to the present invention;

FIG. 11B is a schematic diagram of an ablated fork obtained through driving the central transducer to produce ultrasounds focusing on a focal zone 3 mm under the skin according to the present invention;

FIG. 11C is a schematic diagram of an ablated fork obtained through driving the side transducer to produce ultrasounds focusing on a focal zone 4.5 mm under the skin according to the present invention;

FIG. 12A is a schematic diagram of steps to perform the focused ultrasound method for dermatological treatment according to an embodiment of the present invention; and

FIG. 12B is a schematic diagram of steps to perform the focused ultrasound method for dermatological treatment according to another embodiment of the present invention.

### DETAILED DESCRIPTION OF THE PREFERRED EMBODIMENT

The purpose, construction, features, functions and advantages of the present invention can be appreciated and understood more thoroughly through the following detailed descriptions with reference to the attached drawings.

In the following, an embodiment is used to describe the various details of the present invention. However, it does not mean that this embodiment represents all the embodiments of the present invention. Other embodiments can be envisaged by people familiar with this field, and thus they all fall into the scope of the present invention.

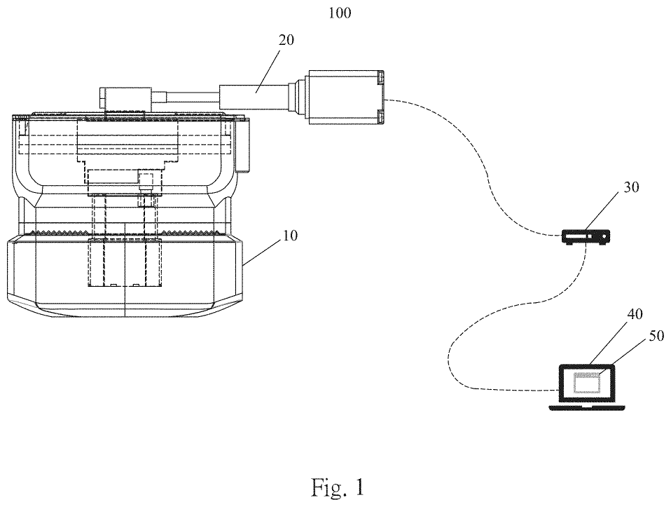

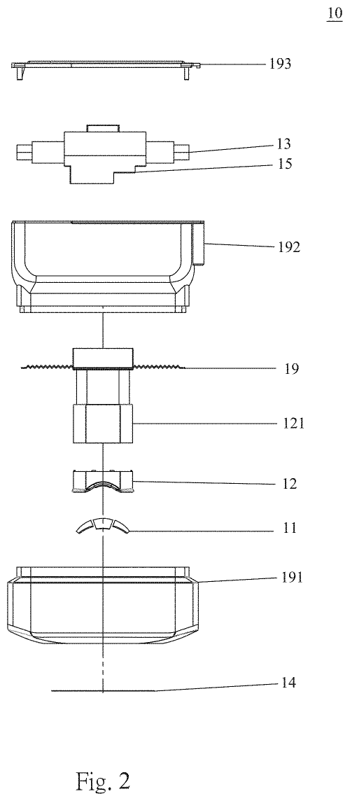

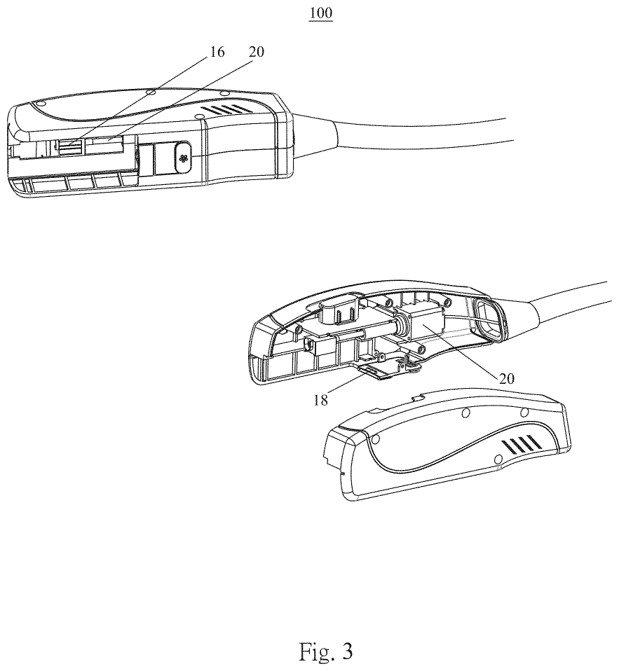

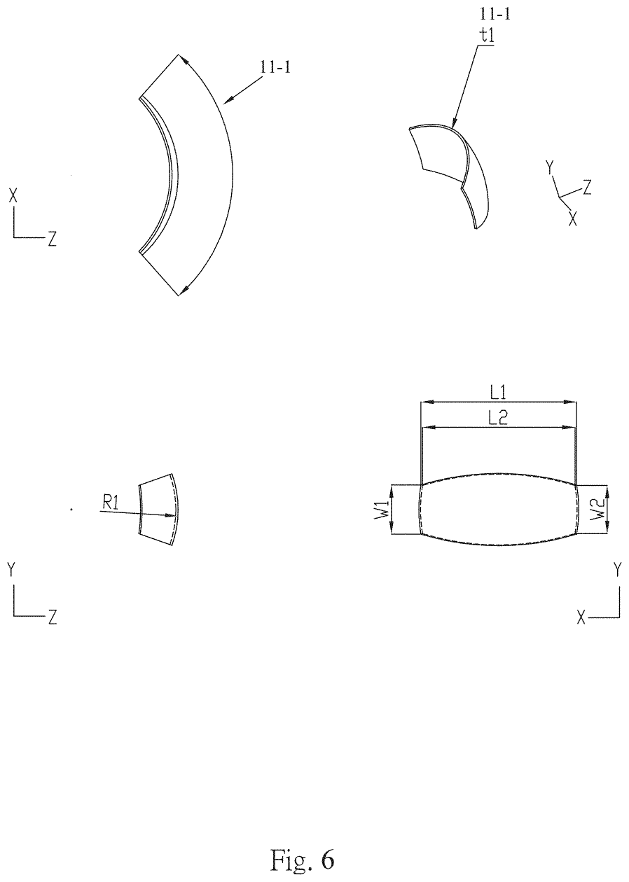

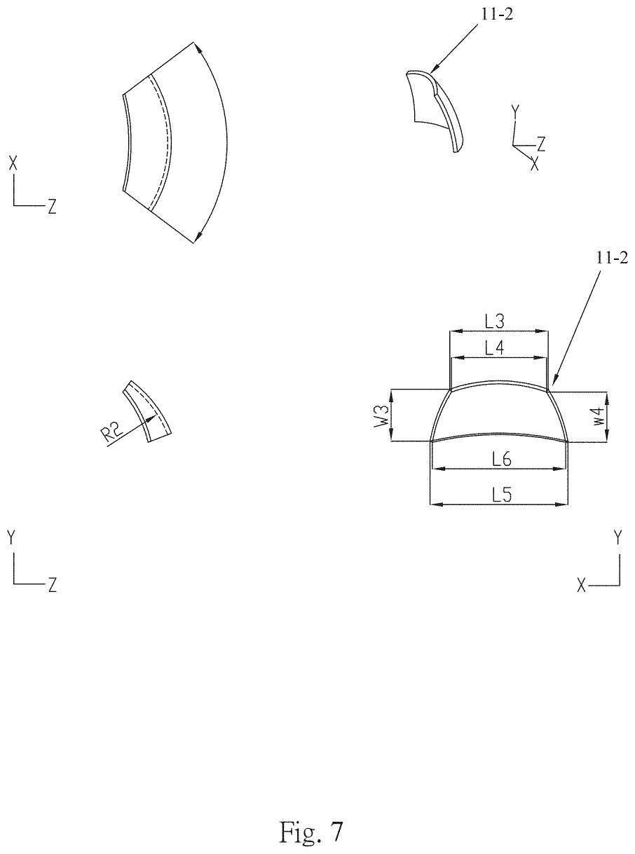

Refer to FIGS. 1 to 12 B respectively for a schematic diagram of a focused ultrasound device for dermatological treatment according to the present invention; a schematic diagram of a focused ultrasound applicator according to the present invention; a schematic diagram of an automatic motion mechanism according the present invention; a schematic diagram of a focused ultrasound applicator and an automatic motion mechanism before and after assembly according to the present invention; a schematic diagram of a focused ultrasound device for dermatological treatment having a central transducer and a side transducer according to the present invention; an exploded view of a central transducer according to the present invention; an exploded view of a side transducer according to the present invention; a schematic diagram showing numerical model of the central transducer and the side transducer in simulation according to the present invention; a diagram of simulation results of focal zone for ultrasound focusing on 3 mm under skin according to the present invention; a diagram of simulation results of focal zone for ultrasound focusing on 4.5 mm under skin according to the present invention; a schematic diagram of a focused ultrasound device used in a scanning type pork ablation experiment according to the present invention; a schematic diagram of an ablated fork obtained through driving the central transducer to produce ultrasounds focusing on a focal zone 3 mm under the skin according to the present invention; a schematic diagram of an ablated fork obtained through driving the side transducer to produce ultrasounds focusing on a focal zone 4.5 mm under the skin according to the present invention; a schematic diagram of steps to perform the focused ultrasound method for dermatological treatment according to an embodiment of the present invention; and a schematic diagram of steps to perform the focused ultrasound method for dermatological treatment according to another embodiment of the present invention.

In order to overcome the shortcomings of the existing technology mentioned above, the present invention provides a focused ultrasound device for dermatological treatment 100, comprising: a focused ultrasound applicator 10, an automatic motion mechanism 20, a driver 30, a computer 40, and a software operation interface 50.

The focused ultrasound applicator 10 is of a cassette type package structure, including: an ultrasound transducer 11, a transducer packaging structure 12, a transmission axle 13, and a sealing film 14 disposed at a front opening of the cassette type package structure, a tenon A 15, a mortise B 16, a tenon C 17, a mortise D 18, a water proof separation layer 194, a transducer cassette upper shell 191, a transducer cassette middle shell 192, and a transducer cassette lower shell 193. The ultrasound transducer 11 is disposed in the transducer packaging structure 12, and the transducer packaging structure 12 is movable linearly along the transmission axle 13. The tenon A 15 is disposed at a top end of the transducer packaging structure 12, and is connected to a mortise B 16 on the transmission axle 13 of a step motor. The sealing film 14 disposed at a front opening of the cassette type package structure is adapted to work in cooperation with the water proof separation layer 194, to tightly seal off water in the cassette type package structure, to serve as a transmission medium of the ultrasound.

For the two sets of geometric parameters mentioned above for the central transducer 11-1 and the side transducer 11-2 respectively, two sets of specific geometric parameters are chosen to perform sound field focusing simulation, to realize tissue ablations at two different depths coaxially, by making use of the focused ultrasound device.

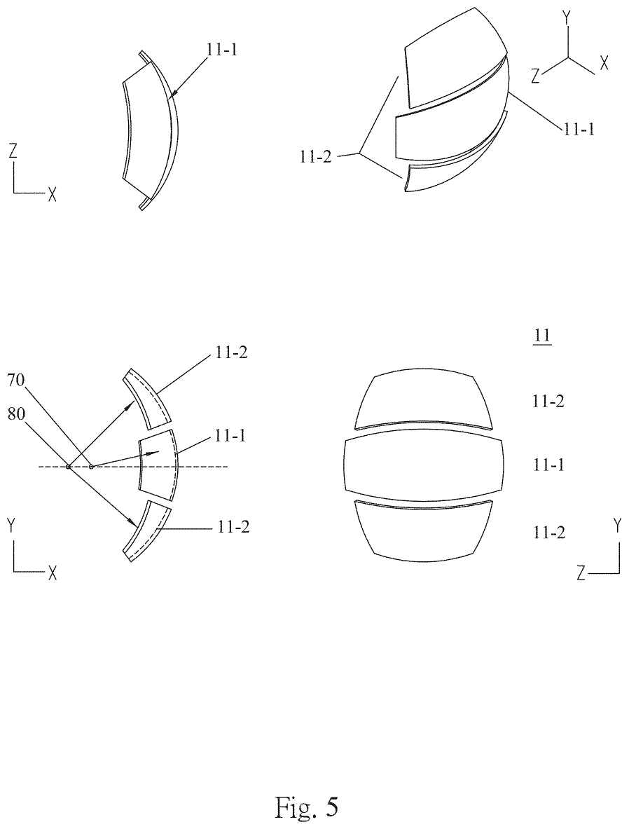

As shown in FIG. 5, the transducer 11 includes a central transducer 11-1 and two side transducers 11-2. The two side transducers 11-2 are of the same geometric structure, and are disposed closely adjacent to the central transducer 11-1. In FIG. 5, the X axis indicates the direction of skin depth. The arrangement of positions for the central transducer 11-1 and two side transducers 11-2 is aimed at producing two focal zones respectively on the same axis, such that the curvature center of the central transducer 11-1 is at the position of the first focal zone 70. Then, arrange the two side transducers 11-2 by using the geometric central line of the central transducer 11-1 as a symmetry axis between the two side transducers 11-2, so that the curvature centers of the two side transducers 11-2 are located coaxially at the position of the second focal zone 80.

In the present invention, two or more frequencies can be used by the driver, to drive two or more transducers to perform ultrasound dermatological ablation at different depths. For example, the driver is able to use the first frequency to drive the central transducer 11-1 to perform ultrasound dermatological ablation at a certain depth. Then, the driver may use the second frequency to drive the side transducer 11-2 to perform ultrasound dermatological ablation at a different depth.

In the present embodiment, the geometric parameters for the central transducer 11-1 are chosen as follows: R 1: 13.5 mm, θ1: 17°, φ1: 95°, t1: 0.3 mm, W1: 2.8 mm, W2: 2.7 mm, L1: 20.5 mm, L2: 20 mm. Besides, the geometric parameters for the side transducers 11-2 are chosen as follows: R2: 15 mm, θ2: 47°, φ2: 73°, t2: 0.5 mm, W3: 10 mm, W4: 9.6 mm, L3: 11.4 mm, L4: 11 mm, L5: 18 mm, L6: 17.8 mm. Then, the sound field focusing simulation for the central transducer 11-1 and the two side transducers 11-2 can be performed by using the Rayleigh-Sommerfeld Diffraction Integral, to calculate the pressure field in the space. Suppose that the surface of the transducer can be divided into infinite number of point sources having area ds, and each point source having a vibration velocity u can be expressed as

μ−μ₀e^{iwt} (1)

μ₀: amplitude of vibration velocity (m/s)
ω: angular frequency (rad/s)
t: time (sec)

Utilize Rayleigh-Sommerfeld Diffraction Integral, to perform integration for the velocity field created by all the point sources of the entire area, to obtain the velocity potential φ_{ρ} for a predetermined point q, as shown as (2)

$${φ}_{p} ( x , y , z ) = \int {\int }_{A} \frac{{ue}^{- jkr}}{2 π r} ds = \int {\int }_{A} \frac{{u}_{0} {e}^{j ( wt - kr )}}{2 π r} ds \qquad ( 2 )$$

r: distance from point source on the transducer to point q
κ: wave number (Wave number, 2π/λ)
λ: wavelength

Then, perform partial differentiation for the velocity potential φ_{ρ} over time, to obtain pressure P:

$$P = j ρ kc \frac{\partial {φ}_{P}}{\partial t} = j ρ kc \int {\int }_{A} \frac{{u}_{0} {e}^{j ( wt - kr )}}{2 π r} ds \qquad ( 3 )$$

ρ: medium density (kg/m³)
c: medium sound velocity (m/s)

The subject obtained in equation (3) is a complex variable, its physical meanings include amplitude of pressure, and the phase of the corresponding point source. Then, perform discretization of the pressure P obtained, to simulate the finite M number of point sources having minute areas obtained by dividing the surface of the transducer, as shown in Equation (4)

$$P = j ρ kc {\sum }_{m = 1}^{M} \frac{{u}_{m} {e}^{i ( wt - {kr}_{m} )}}{2 π {r}_{m}} Δ {S}_{m} \qquad ( 4 )$$

ΔSₘ: area of the m^{th} point source
μₘ: positive vibration velocity of the m^{th} point source
rₘ: distance from the m^{th} point source to a predetermined point

After calculating to obtain the pressure P of ultrasound at point q in the medium, obtain the corresponding Intensity I at point q by using Equation (5).

$$I - \frac{{P}^{2}}{2 ρ c} \qquad ( 5 )$$

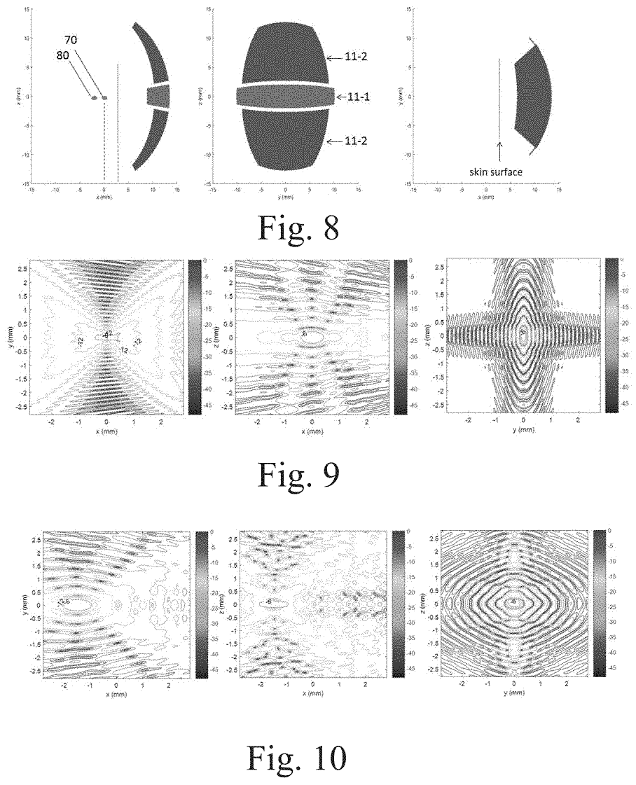

In the descriptions above, program language matlab, and more specifically program language matlab 2015 is utilized in the simulation, to perform modeling of the transducers, and the writing and execution of the sound field simulation program. Moreover, in application, a software Labview is used to run the software operation interface 50. As shown in FIG. 8, in simulation, water is used as a medium for transmitting the ultrasound, with water sound velocity 1500 m/s², water density 1000 kg/m³, and attenuation coefficient 0. As shown in FIG. 8, the numerical model of the central transducer and the side transducer are presented. The X axis indicates the direction of depth that can be reached by the ultrasound of the transducer. Along the X axis, the position having a certain distance to the origin indicates the focal zone of the central transducer, and that is the position of skin at X axis +3 mm. Therefore, the greater the negative value on the X axis, the deeper the ultrasound into the skin. As shown in FIG. 9, the sound field simulation result indicates that, the central transducer could produce a 0.8 mm×0.2 mm×0.4 mm focal zone, 3 mm under the skin. The focal zone is defined as the area enclosed by Intensity peak value of −6 B. Further, as shown in FIG. 10, the sound field simulation result indicates that, the two side transducers could produce a 1.4 mm×0.8 mm×0.4 mm focal zone, that is deeper into the skin, and is 1.5 mm deeper than the focal zone of the central transducer.

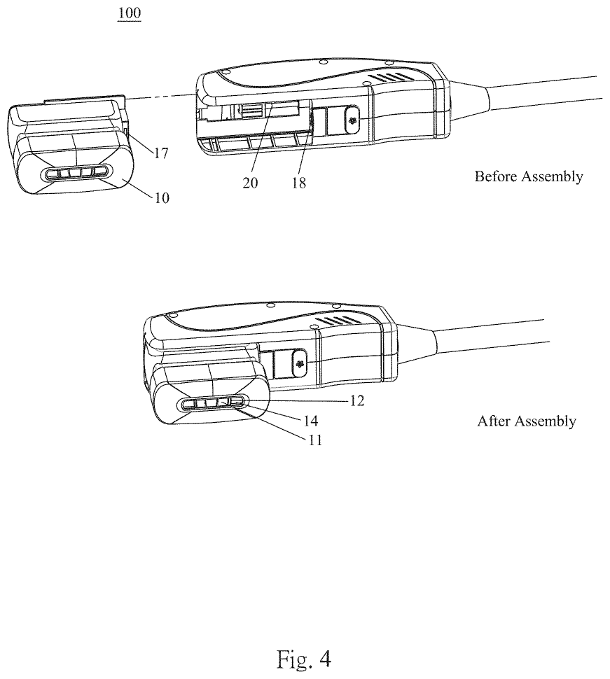

In the present invention, the automatic motion mechanism can be disposed in a hand-held type shell, and it includes a step motor, a transmission axle, a mortise B, a fast detachable button. As shown in FIG. 4, the focused ultrasound applicator can be pushed into the hand-held type shell along a slot in the shell, until the tenon C on the cassette type packaging structure is fixed and locked onto a mortise D on the hand-held type shell, to complete the assembly of the focused ultrasound device. Further, through pushing and pressing the fast detachable button, the cassette type applicator can be detached from the hand-held type shell, and it can be pulled out along the slot, in achieving fast detaching and replacing of the applicator. Through the software operation interface, the focused ultrasound applicator is able to control the transducers, the driver, and the step motor, to perform scanning ablations of target tissues at two different depths.

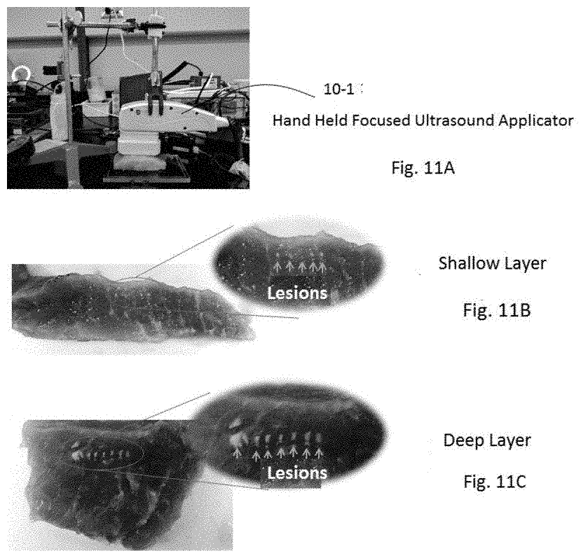

Based on the descriptions above, the Applicant have designed a prototype of a focused ultrasound device to perform the pork scanning ablation experiment, and the experiment setup is as shown in FIG. 11A. In the experiment, the central transducer is driven to produce ultrasound, to be focused on the target tissue 3 mm below the pork skin for a duration of 0.5 second, to complete first focal point pork ablation. Then, as shown in FIG. 11B, control the step motor to bring the central transducer to 5 subsequent points spaced by 3.5 mm respectively, to perform a total of 5 focused ultrasound pork ablations, to cause the portion ablation part to produce white lesions of protein denaturation due to high temperature, while the lesions are located with equal spacing. Finally, as shown in FIG. 11C, on a different location of treatment, and at a position 4.5 mm under the pork skin, perform the target tissue ablation experiment, that includes 7 focused ultrasound ablations, with the former 6 ablations each having duration of 0.5 second, and with the last ablation having duration of 2 seconds, while the transducer is moved a distance of 2.5 mm for each of the ablations. The ablation results show the lesions are disposed with equal spacing.

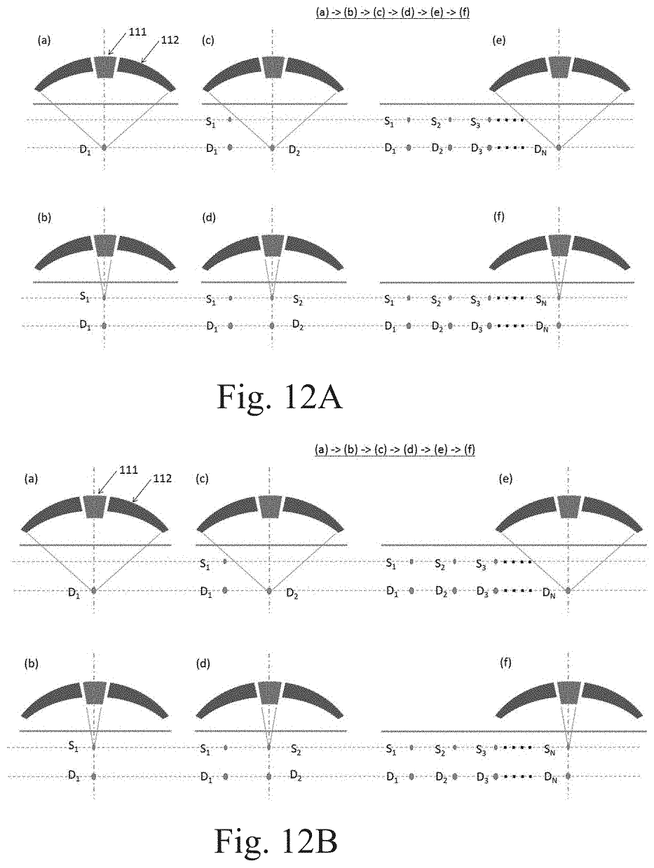

Moreover, refer to FIG. 12A, wherein (a)→(b)→(c)→(d)→(e)→(f) indicates sequence of ablations for easy apprehension. The present invention also provides a focused ultrasound method for dermatological treatment, and that utilizes the focused ultrasound device for dermatological treatment mentioned above to implement. Wherein, a same focused ultrasound applicator is used to perform scanning type ablation coaxially for the deep layer and the shallow layer skin tissues. The method comprises the following steps:

(1) utilize the driver to drive the side transducers in the focused ultrasound applicator, to perform ablation of deep layer target tissues D₁;

(2) shut down the driver, then activate and utilize the driver to drive the central transducer, to perform coaxially ablation of shallow layer target tissues S₁;

(3) utilize the automatic motion mechanism to bring the focused ultrasound applicator to a next treatment position, to repeat driving and ablating of the steps 1 and 2 above, to realize ablation of the deep layer target tissues D₂ and the shallow layer target tissues S₂; and

(4) repeat the steps 1 to 3, to perform in sequence driving, motion, and ablation, to realize coaxial ablation of the deep layer target tissues D₃ . . . D_{N} and the shallow layer target tissues S₃ . . . S_{N}.

Further, refer to FIG. 12B, wherein (a)→(b)→(c)→(d)→(e)→(f) indicates sequence of ablations for easy apprehension. The focused ultrasound method for dermatological treatment can also be implemented in the following steps

(1) utilize the driver to drive the side transducers to act, and control the automatic motion mechanism to move, to perform scanning ablation of the deep layer target tissues D₁, D₂, D₃, . . . D_{N}; and

(2) shut down the driver, and then activate and utilize the driver to drive the central transducer, to perform scanning ablation of the shallow layer target tissues S₁, S₂, S₃, . . . S_{N}, to realize coaxial ablation of skin.

Through using the focused ultrasound device and method for dermatological treatment described above, the deficiencies and drawbacks of the existing technology can be avoided. Namely, the drawback of the existing technology is that, due to geometric limitation and the focal zone high heat requirement, the annular phased array focused ultrasound applicator is not able to perform dynamic focusing to achieve facial skin treatment effectively. In contrast, in the present invention, through the geometric structure design, the transducer is designed into a central transducer and a side transducer, such that they are able to produce focal zones at different depths along the same axis of the focused ultrasound applicator to realize ablation, in achieving skin treatment of the various parts, including the facial part of the body effectively.

The above detailed description of the preferred embodiment is intended to describe more clearly the characteristics and spirit of the present invention. However, the preferred embodiments disclosed above are not intended to be any restrictions to the scope of the present invention. Conversely, its purpose is to include the various changes and equivalent arrangements which are within the scope of the appended claims.

What is claimed is:

1. A focused ultrasound device for dermatological treatment, comprising: a focused ultrasound applicator, an automatic motion mechanism, a driver, a computer, and a software operation interface;
wherein,
the focused ultrasound applicator is of a cassette type package structure, including:
an ultrasound transducer, a transducer packaging structure, a transmission axle, a sealing film disposed at a front opening of the cassette type package structure, a tenon A, a mortise B, a tenon C, a mortise D, a water proof separation layer, a transducer cassette upper shell, a transducer cassette middle shell, and a transducer cassette lower shell;
the ultrasound transducer is disposed in the transducer packaging structure, and the transducer packaging structure is movable linearly along the transmission axle;
the tenon A is disposed at a top end of the transducer packaging structure, and is connected to the mortise B on the transmission axle of a step motor; and
the sealing film disposed at the front opening of the cassette type package structure is adapted to work in cooperation with the water proof separation layer, to tightly seal off the water in the cassette type package structure, with the water serving as a transmission medium of ultrasounds.

2. The focused ultrasound device for dermatological treatment as claimed in claim 1, wherein the ultrasound transducer includes a central transducer and two side transducers, the two side transducers are of a same geometric structure, and are disposed closely adjacent to the central transducer, an arrangement of positions for the central transducer and the two side transducers is aimed at producing two focal zones respectively on a same axis, such that a curvature center of the central transducer is at a position of a first focal zone, and the two side transducers are arranged by using a geometric central line of the central transducer as a symmetry axis between the two side transducers, such that curvature centers of the two side transducers are located coaxially at a position of a second focal zone.

3. The focused ultrasound device for dermatological treatment as claimed in claim 2, wherein a geometric structure of the central transducer is of a concave shell body symmetric in left and right directions, and in up and down directions, and geometric parameters of the central transducer are as follows: a curvature radius R1, a bend polar angle θ1, a bend azimuth angle φ1, a shell thickness t1, an outer width W1, an inner width W2, an outer length L1, and an inner length L2; and ranges of the geometric parameters for the central transducer are as follows: the curvature radius R 1: 10-20 mm, the bend polar angle θ1: 15°-45°, the bend azimuth angle φ1: 80°-120°, the shell thickness t1: 0.2 mm-0.5 mm, the outer width W1: 2 mm-7 mm, the inner width W2: 2 mm-7 mm, the outer length L1: 18 mm-22 mm, and the inner length L2: 18 mm-22 mm.

4. The focused ultrasound device for dermatological treatment as claimed in claim 2, wherein the geometric structure of the side transducer is of a concave shell body symmetric in left and right directions, and in up and down directions, and geometric parameters of the side transducer are as follows: a curvature radius R2, a bend polar angle θ2, a bend azimuth angle φ2, a shell thickness t2, an outer width W3, an inner width W4, an upper outer length L3, an upper inner length L4, a lower outer length L5, and a lower inner length L6; and ranges of geometric parameters of the side transducer are as follows: the curvature radius R 2: 10-20 mm, the bend polar angle θ2: 30°-50°, the bend azimuth angle φ2: 70°-120°, the shell thickness t2: 0.2 mm-0.7 mm, the outer width W3: 6 mm-10 mm, the inner width W4: 6 mm-10 mm, the upper outer length L3: 10 mm-13 mm, the upper inner length L4: 10 mm-13 mm, the lower outer length L5: 16 mm-20 mm, and the lower inner length L6: 16 mm-20 mm.

5. The focused ultrasound device for dermatological treatment as claimed in claim 3, wherein the geometric parameters of the central transducer are chosen as follows: the curvature radius R1: 13.5 mm, the bend polar angle θ1: 17°, the bend azimuth angle φ1: 95°, the shell thickness t1: 0.3 mm, the outer width W1: 2.8 mm, the inner width W2: 2.7 mm, the outer length L1: 20.5 mm, and the inner length L2: 20 mm.

6. The focused ultrasound device for dermatological treatment as claimed in claim 4, wherein the geometric parameters of the side transducers are chosen as follows: the curvature radius R 2: 15 mm, the bend polar angle θ2: 47°, the bend azimuth angle φ2: 73°, the shell thickness t2: 0.5 mm, the outer width W3: 10 mm, the inner width W4: 9.6 mm, the upper outer length L3: 11.4 mm, the upper inner length L4: 11 mm, the lower outer length L5: 18 mm, and the lower inner length L6: 17.8 mm.

7. A focused ultrasound method for dermatological treatment utilizing the focused ultrasound device for dermatological treatment as claimed in claim 1, wherein a same focused ultrasound applicator is used to perform scanning type ablation coaxially for deep layer and shallow layer skin tissues, comprising the following steps:
(1) utilizing the driver to drive the side transducers in the focused ultrasound applicator, to perform ablation of deep layer target tissues D₁;
(2) shutting down the driver, then activating and utilizing the driver to drive the central transducer, to perform coaxial ablation of shallow layer target tissues S₁;
(3) utilizing the automatic motion mechanism to bring the focused ultrasound applicator to a next treatment position, to repeat driving and ablating of the steps 1 and 2 above, to realize ablation of the deep layer target tissues D₂ and the shallow layer target tissues S₂; and
(4) repeating the steps 1 to 3, to perform in sequence driving, motion, and ablation, to realize ablation of the deep layer target tissues D₃ . . . D_{N} and the shallow layer target tissues S₃ . . . S_{N}.

8. The focused ultrasound method for dermatological treatment as claimed in claim 7, comprising the following steps:
(1) utilizing the driver to drive the side transducers to act, and control the automatic motion mechanism to move, to perform scanning ablation of the deep layer target tissues D₁, D₂, D₃, . . . D_{N}; and
(2) shutting down the driver, and then activating and utilizing the driver to drive the central transducer, to perform scanning ablation of the shallow layer target tissues S₁, S₂, S₃, . . . S_{N}, to realize coaxial ablation of skin.
